(19) United States
(12) Patent Application Publication
(10) Pub. No.: US 2015/0335453 A1
(43) Pub. Date: Nov. 26, 2015

(54) Treatment for Obesity by Selectively Clipping the Gastric Fundus to Modify Blood Flow

(71) Applicant: EndoBar Solutions LLC, Orangeburg, NY (US)

(72) Inventors: Nickolas Kipshidze, New York, NY (US); Zurab Kakabadze, Tbilisi (GE)

(73) Assignee: EndoBar Solutions LLC, Orangeburg, NY (US)

(21) Appl. No.: 14/716,345

(22) Filed: May 19, 2015

Related U.S. Application Data
(60) Provisional application No. 62/001,383, filed on May 21, 2014.

(51) Int. Cl. A61F 5/00; A61B 17/128; A61B 17/08

(52) U.S. Cl. CPC A61F 5/0086; A61B 17/083; A61B 17/1285; A61B 2017/00818

(57) ABSTRACT

Weight loss can be promoted by folding a portion of the gastric fundus of a human subject's stomach in proximity to the distal portion of the subject's left gastric artery (including its smaller branches), and clipping the tissue so that it stays in the folded state. The clips cause endovascular flow reduction or interruption to the fundus. The reduced or interrupted blood flow decreases the production of the hormone ghrelin, which reduces the subject's appetite, thereby promoting weight loss. A similar procedure may also be implemented in portions of the subject's stomach that are in proximity to other arteries.

### CROSS REFERENCE TO RELATED APPLICATIONS

This application claims the benefit of U.S. Provisional Application 62/001,383, filed May 21, 2014, which is incorporated herein by reference in its entirety.

### BACKGROUND

Obesity is widely recognized as a major public health issue resulting in decrease of quality of life and development of chronic diseases, such as metabolic syndrome, diabetes, hypertension, congestive heart failure, atherosclerosis, sleep apnea, etc. Lifestyle changes can be used to treat obesity, but lifestyle changes are not always achievable, especially in long term prospect. Drug therapy is one conventional treatment for obesity, but it is often accompanied by various complications and adverse side effects.

Bariatric surgery is another conventional treatment for obesity. One of the recognized benefits of bariatric surgery is the decreased production of ghrelin. Ghrelin, a neuropeptide which is predominantly produced in the gastric fundus, is the only known hormone that stimulates food intake (orexigenic hormone). It is believed that the decreased production of ghrelin that is associated with bariatric surgery helps promote weight loss. But bariatric surgery is invasive and can be accompanied by considerable surgical complications and/or adverse side effects.

### SUMMARY OF THE INVENTION

One aspect of the invention is directed to a method of promoting weight loss. This method includes folding a healthy and non-bleeding portion of a mammal subject's gastric fundus in proximity to the distal portion of the subject's left gastric artery. This method also includes securing the folded portion of the subject's gastric fundus using at least one fastener, wherein the at least one fastener is configured to squeeze the folded portion of the subject's gastric fundus with sufficient force to result in a reduction or interruption of blood flow to a portion of the gastric fundus that is ordinarily supplied with blood by the left gastric artery.

This method may be implemented in a human subject. In some embodiments, the at least one fastener is configured to promote thrombosis in the vicinity of the distal portion of the subject's left gastric artery. In some embodiments, the folding step comprises inwardly folding the portion of the subject's gastric fundus. In some embodiments, the folding step is implemented using endoscopic pliers. In some embodiments, the at least one fastener comprises a plurality of clips, and in some embodiments, these clips are positioned between 1 and 1.5 cm apart from each other. In some embodiments, the at least one fastener is introduced into the subject's stomach via a gastroscope. In some embodiments, the at least one fastener is introduced into the subject's stomach using a laparoscopic approach.

In some embodiments, the at least one fastener includes a coating that comprises a pharmaceutical that helps to decrease production of ghrelin. In some embodiments, the at least one fastener includes a coating that comprises a genetic material that helps to decrease production of ghrelin. In some embodiments, the at least one fastener includes a coating that comprises cells that help to decrease production of ghrelin. In some embodiments, the at least one fastener includes a coating that comprises a drug that helps to decrease or abolish inflammation around the at least one fastener.

In some embodiments, the mammal subject is a human subject, the folding step comprises inwardly folding the portion of the subject's gastric fundus, and the at least one fastener comprises a plurality of clips. These clips may be introduced into the subject's stomach via a gastroscope.

Another aspect of the invention is directed to a method of modifying blood flow to a portion of a mammal subject's stomach that is ordinarily supplied with blood by a given artery. This method includes the step of folding a healthy and non-bleeding portion of a mammal subject's stomach in proximity to the given artery. It also includes the step of securing the folded portion of the subject's stomach using at least one fastener. The at least one fastener is configured to squeeze the folded portion of the subject's stomach with sufficient force to result in a reduction or interruption of blood flow to the portion of the subject's stomach that is ordinarily supplied with blood by the given artery.

In some embodiments, the mammal subject is a human subject. In some embodiments, the folding step comprises inwardly folding the portion of the subject's stomach. In some embodiments, the at least one fastener includes a plurality of clips. In some embodiments, the plurality of clips are positioned between 1 and 1.5 cm apart from each other. In some embodiments, the at least one fastener is introduced into the subject's stomach via a gastroscope. In some embodiments, the at least one fastener is introduced into the subject's stomach using a laparoscopic approach. In some embodiments, the given artery comprises a right gastric artery, a short gastric artery, a gastro-epiploic artery, right gastro-omental artery, a left gastro-omental artery, or any branches thereof. In some embodiments, the mammal subject is a human subject, the folding step comprises inwardly folding the portion of the subject's stomach, and the at least one fastener comprises a plurality of clips.

Another aspect of the invention is directed to a clip for clipping a portion of a human stomach. This clip includes a first section having a proximal portion and a set of distal jaws, with an inversion region disposed between the proximal portion and the set of distal jaws. The first section is configured from a spring material so that when the proximal portion is squeezed together, the distal jaws are forced apart, and when the proximal portion is released, the distal jaws automatically return towards other by spring action. The clip also includes a U-shaped section having two arms, with a distal end of each arm connected to a distal end of a respective one of the distal jaws. The U-shaped section is configured to exert force in order to return to its original U shape.

In some embodiments, the clip includes a ridged surface provided at an inner edge of the U shaped section. In some embodiments, the U-shaped section is made from a shape memory material. In some embodiments, the U-shaped section is made from a spring material. In some embodiments, the clip has an overall length between 4.5 and 5.5 cm, and the distal jaws have a length between 3 and 5 cm. In some embodiments, the jaws are spaced between 10 and 15 mm apart in a closed position and the jaws are spaced between 13 and 19 mm apart in an open position. In some embodiments, the first section comprises at least one of titanium and stainless steel, and wherein the U-shaped section comprises nitinol.

In some embodiments, the clip has a ridged surface provided at an inner edge of the U shaped section, and the clip has an overall length between 4.5 and 5.5 cm. The distal jaws have a length between 3 and 5 cm, and the jaws are spaced between 10 and 15 mm apart in a closed position and spaced between 13 and 19 mm apart in an open position. The first section comprises at least one of titanium and stainless steel, and the U-shaped section comprises nitinol.

### BRIEF DESCRIPTION OF THE DRAWINGS

FIG. 1A is an exemplary drawing of an external view of a subject's stomach including the left gastric artery.

FIG. 1B is an exemplary drawing of an external view of a subject's stomach including the left gastric artery, where a portion of the left gastric artery has been captured in a fold of the gastric fundus.

FIG. 2 depicts an example of a suitable clip for introduction into the gastric fundus.

FIG. 3A is a magnified gastroscopic image of the fundus of a subject pig prior to performing the folding and clipping procedure.

FIG. 3B is a gastroscopic image of the fundus of a subject pig with clips placed in the fundus.

FIG. 3C is a magnified gastroscopic image of the fundus of a subject pig after the folding and clipping procedure was performed.

FIG. 4 is a graph that shows how the weight of the subject pigs changed over time.

FIG. 5 is a graph that shows how the ghrelin level in the subject pigs' blood changed over time.

FIG. 6A is a histologic section image of the mucosa in the area of the left gastric artery in the stomach of a subject pig, after the folding and clipping procedure was performed.

FIG. 6B is a magnified view of a portion of the FIG. 6A image.

FIG. 7A is a histologic section image of the mucosa in the area of the left gastric artery in the stomach of a control pig, in which the folding and clipping procedure was not performed.

FIG. 7B is a magnified view of a portion of the FIG. 7A image.

FIG. 8A is a diagram depicting an exemplary invagination of the fundus of the stomach of a subject rat.

FIGS. 8B and 8C are images of the fundus of the stomach of a subject rat, after invagination and clipping was performed.

FIG. 9A is an image of the left gastric artery of the stomach of a subject rat, taken after the folding and clipping procedure was performed.

FIG. 9B is an image of the left gastric artery of the stomach of a subject rat, taken before the folding and clipping procedure was performed.

FIG. 10 depicts a clip designed for performing clipping of the stomach fundus in human subjects.

### DETAILED DESCRIPTION OF THE PREFERRED EMBODIMENTS

Gastroscopic modification of the function of the gastric fundus using one or more fasteners (e.g., clips) to promote thrombosis in the vicinity of the distal portion of the left gastric artery is less invasive and more cost effective alternative to bariatric surgery for achieving weight loss.

This application describes a novel approach which involves modifying the arterial blood flow to the gastric fundus by means of gastroscopic clipping of the fundus to promote blood flow reduction (or interruption) to the fundus. The procedure is preferably implemented on a healthy and non-bleeding portion of a mammal subject's gastric fundus in proximity to the distal portion of the subject's left gastric artery. Experiments in pigs have demonstrated dramatic weight loss at one month after procedure with no reported adverse effects. While reduction in the hunger-mediating peptide hormone ghrelin (secreted in the gastric fundus) has been identified as one possible mechanism, the complete physiologic mechanism is not yet clear and may well involve other hormones and/or changes in gastric motility with consequent reduction in hunger sensation in experimental subjects.

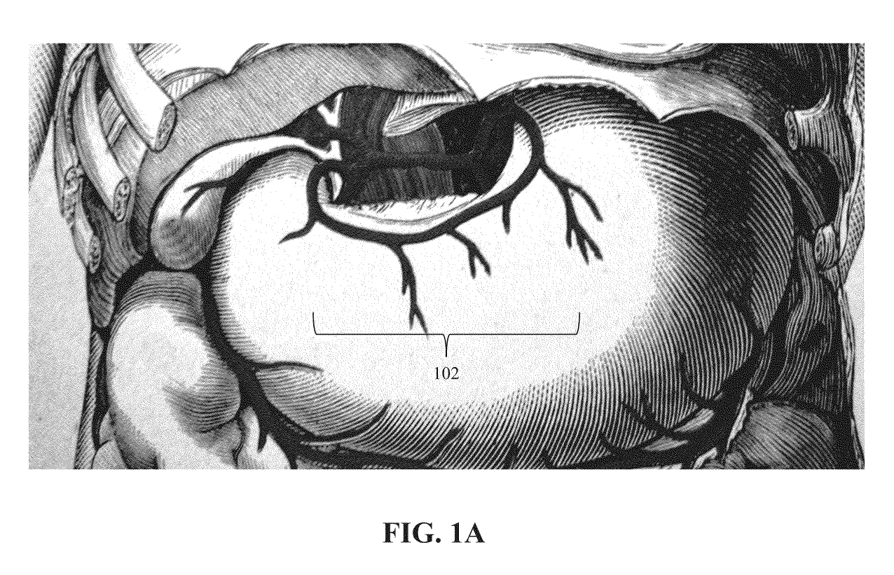

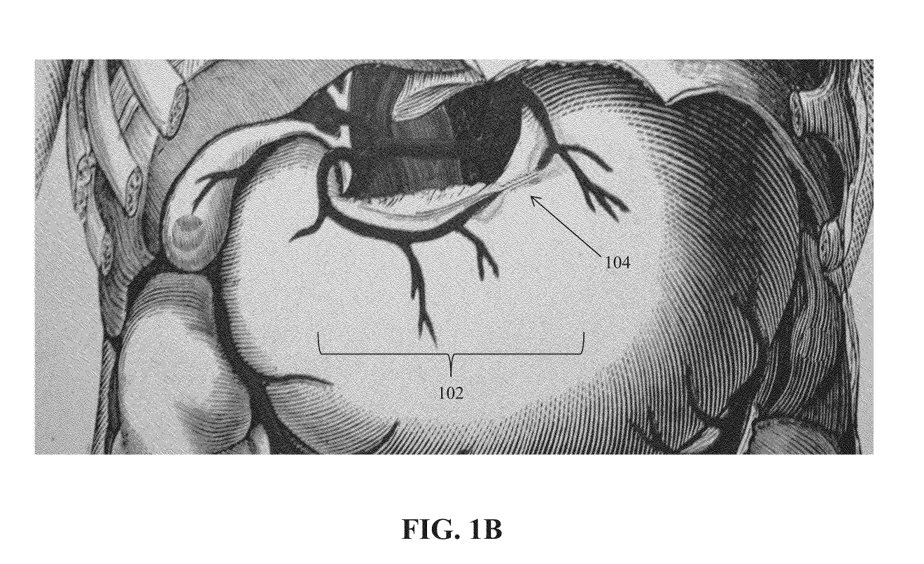

FIG. 1A is a drawing of an external view of a subject's stomach including the left gastric artery 102. As shown in FIG. 1A, the left gastric artery 102 is positioned along the top side of the stomach.

FIG. 1B is an exemplary drawing of an external view of a subject's stomach including the left gastric artery 102, where a portion 104 of the left gastric artery has been captured in a fold of the gastric fundus using the procedure described herein to clip a portion of the gastric fundus in proximity to the left gastric artery.

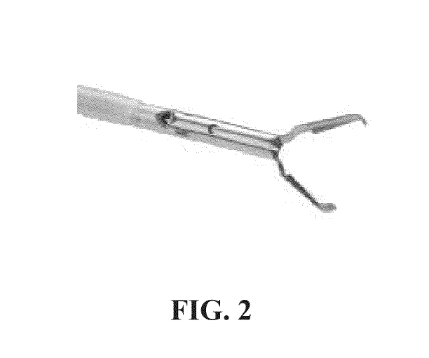

The approach described herein achieves endovascular flow reduction or interruption using at least one fastener to clamp a portion of the gastric fundus in the vicinity of the left gastric artery. In some preferred embodiments, this is implemented by introducing two or more clips into an area of the gastric fundus in proximity to the distal portion of the subject's left gastric artery. FIG. 2 depicts an example of a suitable clip for introduction into the gastric fundus for this purpose. In some embodiment, these clips have a clip opening of between 7.5 mm and 30 mm, with a preferred opening of between 20 to 25 mm. The clips can have an opening angle of between 90 degrees and 135 degrees. The clips are preferably delivered to the subject's stomach via a gastroscope. The clips are attached to a cable within a sheath, enclosed within a delivery/deployment catheter. The cable/catheter can have a working length of between 1550 mm and 2700 mm, and a minimum working channel of 2.8 mm. Example suitable commercially available products for this purpose include: the Resolution® Clip (Boston Scientific Corp., Natick, Mass.); the QuickClip2 and EZClip (Olympus America, Inc., Center Valley, Pa.); the Over-The-Scope Clip® System (Ovesco Endoscopy AG, Tubingen, Germany); and the Instinct™ Endoscopic Hemoclip (Cook Medical Inc., Bloomington, Ind.).

In alternative embodiments, other fasteners may be used, including but not limited to staples, clamps, sutures, and any other material that can hold or bind tissue to occlude a blood vessel and promote thrombosis.

Optionally, the clips (or other fasteners) may be coated with certain substances to enhance the effect of the procedure. Examples include, but are not limited to: pharmaceuticals, genetic materials, or different types of cells that also help to decrease production of ghrelin and/or other hormones or other substances that effect appetite in humans.

The procedure preferably involves inserting a gastroscope into the subject's stomach. After the gastroscope is in position, one or more fasteners (e.g., 2 clips) are then guided through the gastroscope and introduced into an area of the gastric fundus in proximity to the mid segment or distal portion of the subject's left gastric artery. In a preferred embodiment, the clips are placed approximately 1 cm apart from each other. In some embodiments, a laparoscopic approach can be used to introduce the clips into the subject's stomach using, e.g., a LIGAMAX™ 5 Endoscopic Clip Applier (Ethicon Endo-Surgery, Inc., Cincinnati, Ohio).

The presence of the clips (or other fasteners) in the gastric fundus in proximity to the distal portion of left gastric artery reduces or interrupts the blood flow to the fundus. More specifically, it reduces or interrupts the blood supply to the fundus by promoting thrombosis in an area around the clips (or other fasteners), typically 2-3 cm around each clip. The thrombosis has the effect of growing into the left gastric artery and thereby reducing or interrupting blood flow. An advantage of the above-described procedure is that the thrombosis and corresponding blood flow reduction occurs even if the clips (or other fasteners) are not placed precisely on the area of the fundus occupied by the left gastric artery. Clips (or other fasteners) placed in proximity to the left gastric artery promote the same thrombosis and blood flow reduction.

### Example 1

A study was done on three pigs to determine the feasibility, safety and efficacy of clipping of the gastric fundus in proximity to the distal portion of the left gastric artery to reduce plasma ghrelin levels and body weight. Two of the pigs underwent the complete procedure including folding and clipping of the gastric fundus, and one pig underwent the complete procedure except for the folding and clipping as a control. Pigs were chosen for the study because of the anatomical similarities with a human's gastric fundus, and gastric and peripheral arteries. Moreover, a pig's stomach is accessible with conventional equipment, such as a gastroscope, which allowed for the evaluation of the placement of the clips.

Each animal's body weight was recorded upon initiation in the study, prior to treatment and at every follow-up until the end of the study. Non-atherosclerotic healthy Yorkshire Domestic Swine (non-specific pathogen-free (SPF) status) of either sex were used for the study. All animals were obtained from an approved vendor and USDA-certified facility that prescreens, with veterinarian oversight, the physical examination of each animal to ensure that only healthy swine were used in the study. The animals were in the weight range of 27-30 kg at the time of receipt and initiation into the study. Each animal was fed accordingly through the course of the study to control the growth rate and satisfy the animal's appetite. Water was available ad-lib.

The animals were monitored and observed approximately three or more days prior to experimental use. The animals were pretreated with aspirin (324 mg=4*81 mg), clopidogrel bisulfate (75 mg; i.e. Plavix) daily for a minimum of three days prior to the study, and were given nifedipine (30 mg; i.e. Procardia XL) the night before the study. The dose of aspirin, clopidogrel bisulfate and nifedipine were given based on the accepted standard of care as reported in the literature and not based on body weight. The pigs were fasted a minimum of 12 hours prior to surgical procedures.

For the study, the pigs underwent an angiography procedure to monitor the blood flow in the area of the gastric fundus and left gastric artery to confirm the efficacy of the clipping in reducing or interrupting blood flow. It should be appreciated that the angiography is not considered part of the inventive weight loss procedure described herein, and the inventive procedure can be performed without an angiography. Surgical sites on the right and left femoral triangle were prepared by clipping the hair, and cleansing the sites with iodine scrub alternating with 70% isopropyl alcohol and painting with iodine solution. An arterial sheath was placed in the access artery using percutaneous insertion or surgical isolation with sterile technique. Selective angiography was completed utilizing standard catheter techniques with diagnostic catheters and micro catheter. Placement of the micro catheters into the arterial segments was delivered over standard guidewires, based on angiographic dimensions using fluoroscopic guidance. Diagnostic angiography was performed using contrast medium in orthogonal views (left anterior oblique (LAO) and right anterior oblique (RAO)) of left gastric arterial segments.

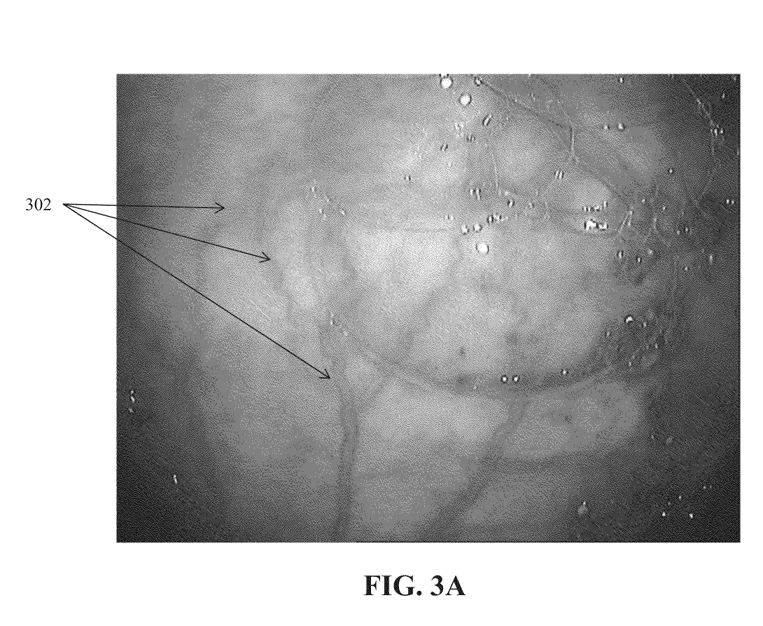

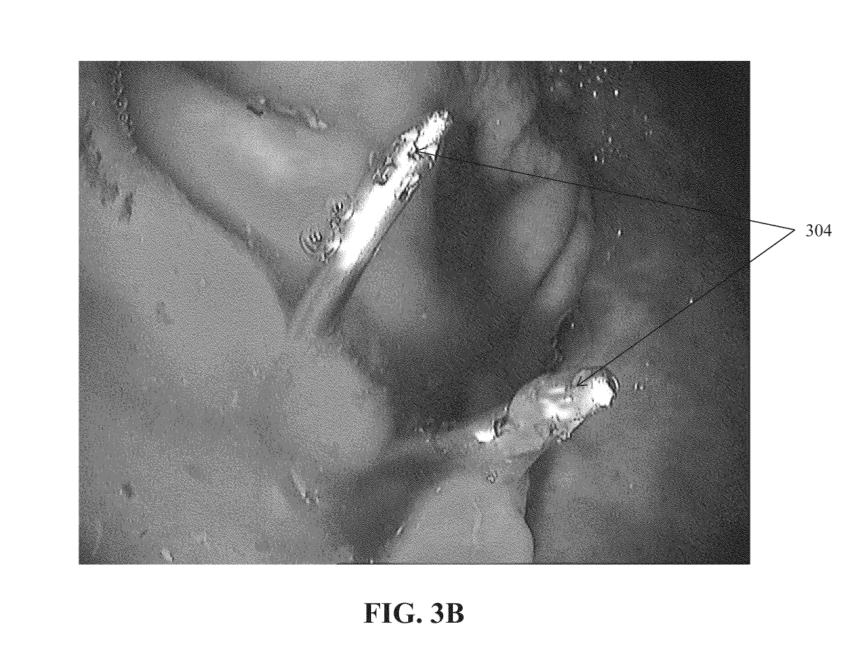

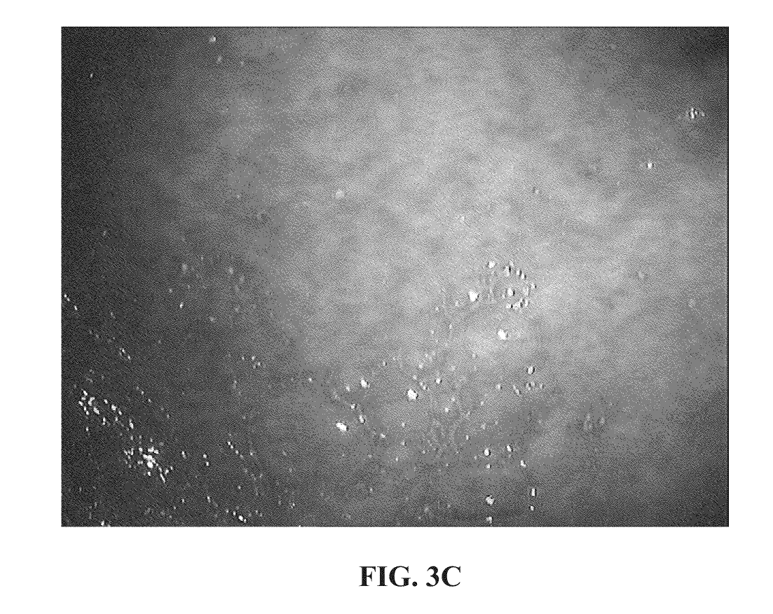

FIG. 3A is a gastroscopic image of the fundus of a subject pig prior to performing the folding and clipping procedure, magnified to show the small vasculature. As shown in FIG. 3A, the fundus exhibits normal vasculature—small vessels (e.g., such as those denoted by reference number 302) are observed in the fundus.

In the inventive weight loss procedure, a gastroscope was advanced into the stomach of the pig. In some embodiments, the gastroscope is equipped with an ultrasound probe. An endoscopic pair of pliers (i.e., an endoscopic grasper) was introduced into the stomach via the gastroscope. The area of the gastric fundus in proximity to the distal portion of the left gastric artery was located, and the pliers were used to inwardly fold a portion of the gastric fundus in proximity to the distal portion of the left gastric artery. As the left gastric artery is not positioned on the surface of the gastric fundus, it is preferred to fold an area of the gastric fundus that measures approximately 4.0 cm in length and approximately 1.0 cm in depth. This may be accomplished, for example, using a pair of clips that are each 4.0 cm long, with the clips spaced between 1 and 1.5 cm apart from each other.

Once the gastric fundus tissue was folded with the pliers, a clip (e.g., a Resolution® Clip available from Boston Scientific Corp., Natick, Mass.) was inserted into the stomach via the gastroscope. The jaws of the clip were opened and the jaws were positioned adjacent to the folded area of the gastric fundus. The clip was then closed on the folded area of the gastric fundus and deployed to hold the folded tissue in place. A second clip was deployed in the folded tissue in the same manner. The second clip was placed approximately 1.0 cm apart from the first clip. While this procedure was performed using two clips, it should be understood that additional clips can be deployed to fold a larger area of the gastric fundus. Preferably, two to four clips are used. In human subjects, more than four clips may be needed. In some embodiments, a second row of clips can be deployed to secure a larger area of folded tissue.

FIG. 3B is a gastroscopic image of the fundus of a subject pig with clips placed in the fundus. As shown in FIG. 3B, two clips 304 are deployed in the fundus to secure a folded portion of the fundus. FIG. 3C is a gastroscopic image of the fundus of a subject pig that was taken about 40 minutes after the folding and clipping procedure was performed, magnified to show the small vasculature. The fundus depicted in FIG. 3C exhibits decreased vasculature with respect to the pre-clipping fundus shown in FIG. 3A. For example, the small vessels 302 observed in FIG. 3A are not visible in FIG. 3C.

After the clips were deployed as described above, the gastroscope was removed from the pig's stomach. Post-treatment angiography was completed utilizing the same diagnostic catheters as previously described. Following treatment, the guidewire and arterial sheath were removed, the skin incision was sewed with suture material and the upper skin layer was closed with the placement of skin staples.

Following the procedure, the animals were closely monitored during anesthetic recovery for physiological disturbances including cardiovascular/respiratory depression, hypothermia, and excessive bleeding from the surgical site. The animals were removed from Isoflurane and were ventilated with room air until breathing on their own. The animals were then returned to their pen where they are monitored until they return to a sternal position.

Blood samples were taken before and at 24, 48 and 78 hours after the procedure. Clotted samples were centrifuged to separate out blood plasma. Fasting levels of ghrelin were measured using the Ghrelin (TOTAL) RIA KIT (Merck Millipore). Weight and fasting plasma ghrelin levels were also obtained at initial baseline and at 1 week, 2 weeks, 3 weeks and 4 weeks post-procedure.

Prior to scheduled euthanization and necropsy, the pigs were sedated with a combination of Ketamine 20 mg/kg; Xylazine 5 mg/kg, and atropine 0.03-0.05 mg/kg by intramuscular injection. An intravenous line was established, and the animals were intubated and ventilated with oxygen (2 L/min), and Isoflurane 0.5-5.0% (2 L/min) administered by inhalation. An arterial sheath was placed in the access artery using percutaneous insertion or surgical isolation with sterile technique. Heparin (5000 Units) was administered via the arterial sheath. A guide catheter was placed into the sheath. Angiographic images were taken with contrast media to evaluate the treatment site within each artery.

The angiograms from the initial procedure and at the study endpoint prior to euthanasia were recorded in a digital format. During initial treatment and at the scheduled follow-up, the left gastric artery was evaluated and graded according to the following parameters: the presence or absence of an aneurysm (greater than 20% enlargement of the artery at the region of the stent), the presence or absence of an intraluminal filling defect, and the patency of the artery were recorded.

The animals were then euthanized per CRI standard operating procedures (Fatal Plus (390 mg/ml pentobarbital sodium; 1% propylene glycol; 29% ethyl alcohol, 2% benzyl alcohol), by IV while under general anesthesia). The treated tissues were excised immediately while keeping the excised vessels intact. The animals were then examined.

The stomachs were opened and fixed in formaldehyde and were send to pathology laboratory. The external aspects of the stomachs were unremarkable. No hemorrhage or perforation is noted. Gross pictures of the stomachs are taken. Twelve tissue sections from the fundus, two tissue sections from the body and two tissue sections from the pyloric regions were taken for routine paraffin embedding and sectioning). Four-micron histologic sections are stained with routine H&E stain and immunohistochemical stain for Ghrelin (Ghrelin [Porcine] antibody for IHC, catalog # H-031-52, Phoenix Pharmaceuticals, Inc., 330 Beach Road, Burlingame, Calif. 94010, USA (800) 988-1205) at 1:2000 dilution. These slides were scanned with a whole-slide scanner using the 20× objective (Aperio ScanScope System, Aperio Technologies, Inc. 1360 Park Center Drive, Vista, Calif. 92081 866-478-4111). Using the Aperio ScanScope software with the Genie and the nuclear algorithms we measured the mucosal area and the number of brown staining Ghrelin positive cells. The algorithm was validated against the manual count with an average discrepancy of 3.05% excluding the three histologic section outliers.

Histopathologic and immune-histopathologic analysis was performed to evaluate overall tissue architecture, ulcerations, damage to gastric mucosa, and viability of parietal cells.

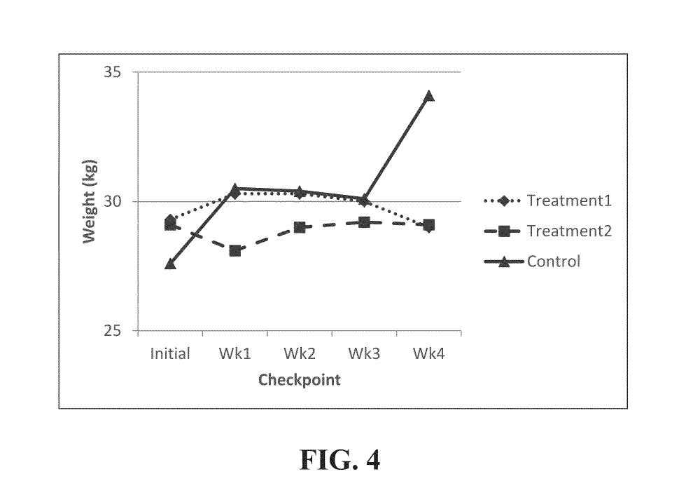

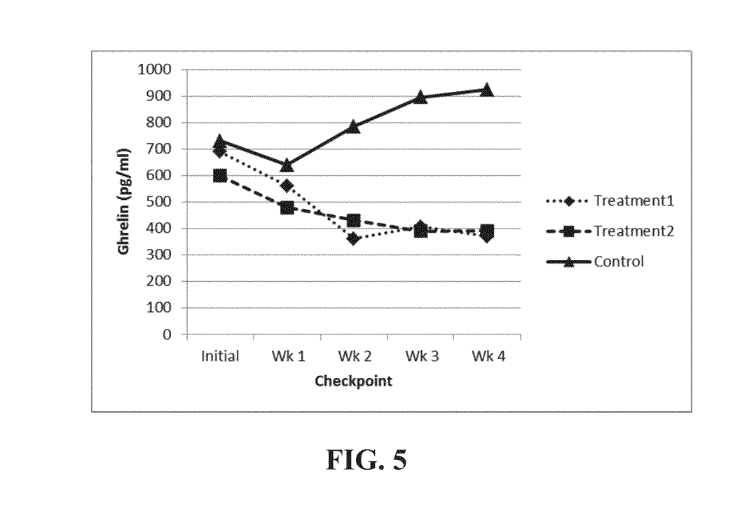

Data for the study is presented below. Table 1 shows the subject animal data, Table 2 shows the weight at each checkpoint for each subject animal and Table 3 shows the ghrelin levels at each checkpoint for each subject animal. FIGS. 4 and 5 depict the data in Tables 2 and 3, respectively, in a graphical format.

TABLE 1

| Parameter | Value |
|---|---|
| Number of animals (pigs) | 3 |
| Weight (kg) | 28.7 ± 0.9 |
| Ghrelin (pg/ml) | 673 ± 67.2 |

The pattern of change in weight over time was significantly different between the control and experimental animals. The average post-procedure weight gain in experimental animals was significantly lower than that in control animals (−0.3 kg versus 6.5 kg, respectively).

TABLE 2

| | | Initial | | | | |
|---|---|---|---|---|---|---|
| Subject | | weight | Weight at | Weight at | Weight at | Weight at |
| Animal # | Type | (kg) | 1 week FU | 2 week FU | 3 week FU | 4 week FU |
| 1 | Treatment | 29.3 | 30.3 | 30.3 | 30.0 | 291.0 |
| 2 | Treatment | 29.1 | 28.1 | 29.0 | 29.2 | 29.1 |
| 3 | Control | 27.6 | 30.5 | 30.4 | 30.1 | 34.1 |

TABLE 3

| | | Initial | | | | |
|---|---|---|---|---|---|---|
| | | Ghrelin | Ghrelin | Ghrelin | Ghrelin | Ghrelin |
| | | level | level at | level at | level at | level at |
| Subject # | Type | (pg/ml) | 1 week FU | 2 week FU | 3 week FU | 4 week FU |
| 1 | Treatment | 689.67 | 560.76 | 360.78 | 407.23 | 370.07 |
| 2 | Treatment | 598.98 | 479.43 | 430.47 | 389.22 | 390.94 |
| 3 | Control | 730.22 | 640.17 | 785.34 | 894.71 | 923.45 |

RESULTS: In the two treated pigs, significant decrease of vascularization in large area of gastric fundus around clips was observed via gastroscopy immediately following the clipping procedure. In addition, the post-procedure angiography demonstrated interruption of blood flow in the distal portion of the left gastric artery.

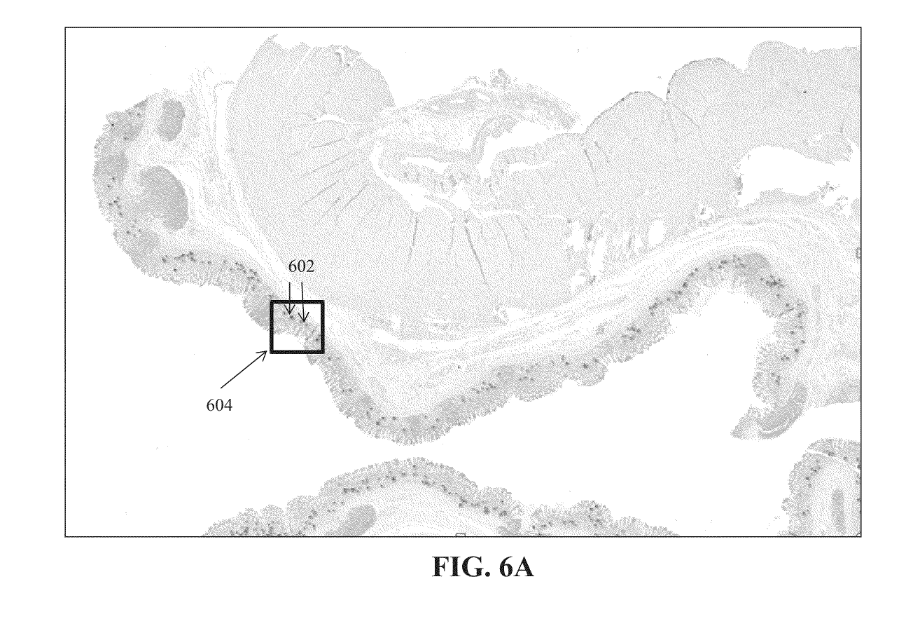

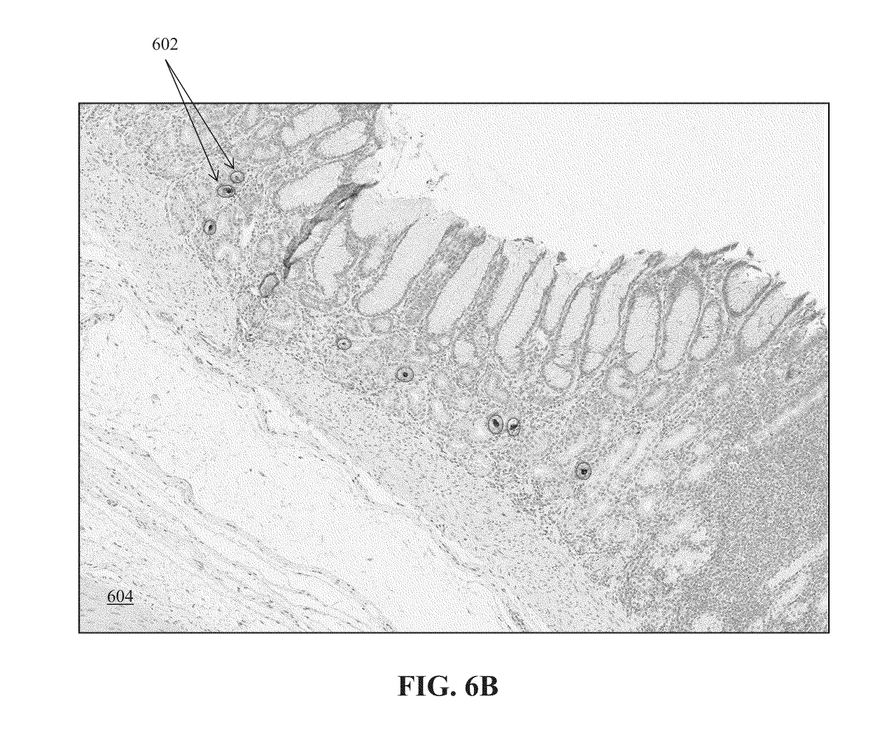

FIG. 6A is a histologic section image, stained with immunohistochemical stain for Ghrelin, of the mucosa in the area of the left gastric artery in the stomach of a subject pig in which the folding and clipping procedure was performed (obtained after the pig was sacrificed). The area of the mucosa shown in FIG. 6A is 8.79 square millimeters. As shown in FIG. 6A, the dark points (e.g., such as denoted by reference number 602) represent ghrelin-positive cells in the mucosa. The box 604 indicates a portion of the mucosa that is magnified in FIG. 6B to show greater detail. The circled areas 602 in FIG. 6B correspond to the ghrelin-positive cells in that portion of the mucosa. The total number of ghrelin-positive cells in FIG. 6A is 166, or 18.89 ghrelin-positive cells per square millimeter.

In contrast, the control pig demonstrated normal appearance of gastric mucosa and no changes to the blood flow in the distal portion of the left gastric artery.

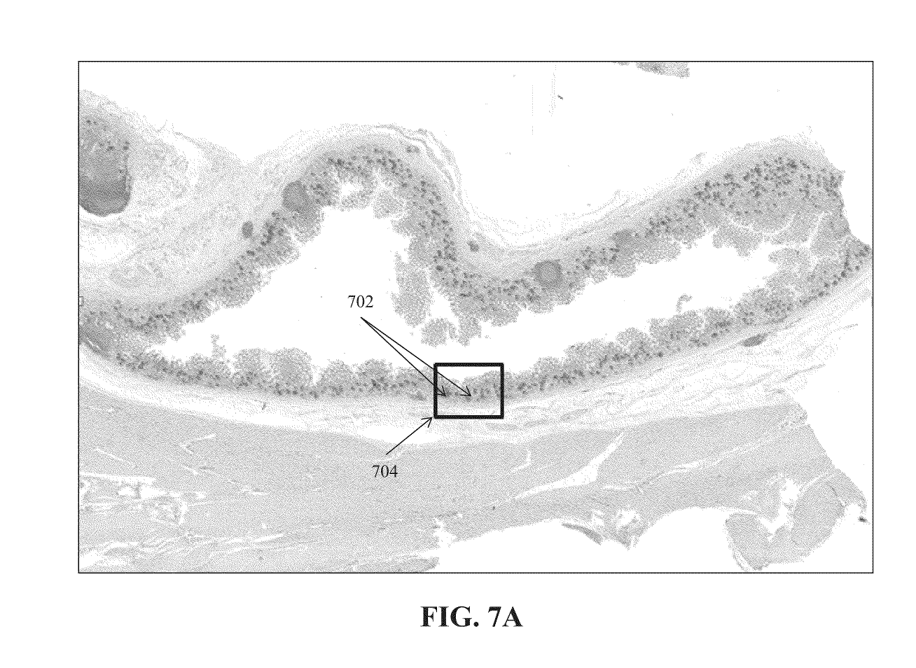

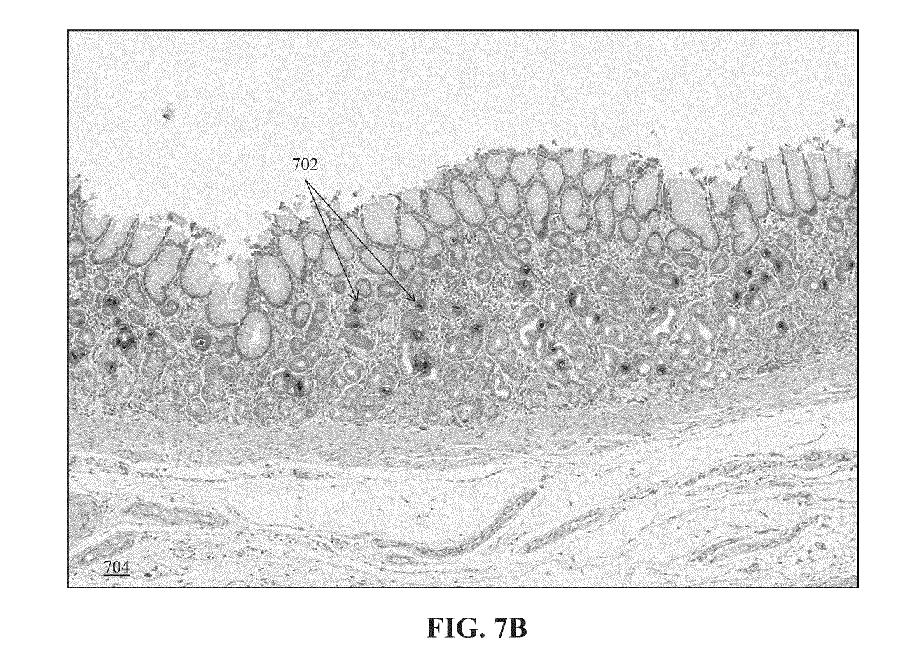

FIG. 7A is a histologic section image, stained with immunohistochemical stain for Ghrelin, of the mucosa in the area of the left gastric artery in the stomach of a control pig, in which the folding and clipping procedure was not performed (obtained after the pig was sacrificed). The area of the mucosa shown in FIG. 7A is 20.87 square millimeters. As shown in FIG. 7A, the dark points (e.g., such as those denoted by reference number 702) represent ghrelin-positive cells in the mucosa. The box 704 indicates a portion of the mucosa that is magnified in FIG. 7B to show greater detail. The circled areas 702 in FIG. 7B correspond to the ghrelin-positive cells in that portion of the mucosa. The total number of ghrelin-positive cells in FIG. 7A is 1,086, or 52.04 ghrelin-positive cells per square millimeter.

As set forth above, ghrelin-immunoreactive cell density was significantly lower in the gastric fundus in the treated animals versus the control animal. Both treated animals demonstrated minimal changes in the mucosa of gastric fundus but there was evidence of marked decrease of vascular bed in the fundal area. This observation confirms that folding and clipping was of adequate extent to induce controlled ischemia.

The data above demonstrates that thrombosis in the vicinity of the distal portion of the left gastric artery using clips is associated with significant reductions in plasma ghrelin levels in pigs. It should be noted, however, that after an initial pronounced decline in ghrelin levels after the procedure, the ghrelin level did increase slightly for each of the treated pigs at the 3 or 4 week follow-up (i.e., the ghrelin level for Treated Pig 1 increased at the 3-week follow-up and the ghrelin level for Treated Pig 2 increased at the 4-week follow-up). Although the levels were still lower than the pre-procedure baseline, a long-term study may be warranted to further investigate this increase.

The procedure described above appears to be safe. Specifically, there were no incidences of ulcer formation or injury to remote structures. There were no sign of perforations or ulcers in all pigs.

In alternative embodiments, different approaches for implementing folding of the fundus before the clips are attached may be used instead of the endoscopic pliers described above. In one example, two pairs of endoscopic pliers are used to implement folding, spaced a few cm apart. The jaws of these pliers may be closed simultaneously or sequentially. After the folding is implemented using both pairs of pliers (each of which is preferably delivered via its own working port in the gastroscope), the clip (which is preferably delivered via a third working port) is applied to the folded region that lies between the two pairs of pliers. This procedure is then repeated for the remaining clips. In another example, one or more hooks are used to grab the tissue and pull the tissue inward, and one or more clips are then applied to the region that is being pulled by the hooks. In yet another example, one or more air-tight tubes designed to resist collapse are delivered via a gastroscope, and a pump is connected to the proximal end of the tubes to evacuate enough air from the tubes to generate suction. When the distal end of each tube is pressed against the inner wall of the stomach, the distal end of the tube will stick to the wall due to the suction. Then, pulling the tube in a proximal direction will pull the tissue inward, and one or more clips are then applied to the region that is being pulled inward by the suction.

In alternative embodiments, a single fastener may be used instead of a plurality of clips. In this situation, the single fastener is preferably configured to squeeze a wide region of the gastric fundus (e.g., 1-2 cm wide) in the vicinity of the left gastric artery.

### Example 2

Studies were also performed in rats. All experiments were performed on ten white laboratory rats of both sexes weighing approximately 250-300 g. The animals were housed in standard laboratory conditions under 12-hour day-night cycles with provision of pelleted rodent diet and water ad libitum. All animals received care according to institutional guidelines. The animals were provided only water for 1 day before surgery.

For the rat study, the following procedure was performed. Three groups of animals were included in the study: one group of ten rats that served as controls, and two groups of ten rats where each group underwent a particular treatment of folding and clipping the fundus of the stomach.

For the control group, a laparotomy was performed on each rat to access the interior of the stomach. Visualization of the left gastric artery with dye was then performed to determine blood flow in the artery.

For the first treatment group, each rat underwent a laparotomy to access the interior of the stomach. Ligation of the left gastric artery with sutures from the parietal side of the stomach was then performed on each rat. Visualization of the left gastric artery with dye was then performed to determine blood flow in the artery.

For the second treatment group, each rat underwent a laparotomy to access the interior of the stomach. A portion of the fundus in proximity to the left gastric artery was then folded inwardly and clipped in place using two to four clipping devices. Visualization of the left gastric artery with dye was then performed to determine blood flow in the artery.

The procedures were carried out under ethyl ether anesthesia mask. After opening the abdominal cavity in the wall of the stomach, the position of the left gastric artery was identified. A small incision of 0.5 cm on the greater curvature was performed, and through this incision a probe was inserted for gastric lavage of food masses. The stomach wall of the mucosa was then invaginated using microsurgical clips so that the intussusceptum would include the left gastric artery—stacked so that the left gastric artery would be between the walls of the stomach. Two clips were placed perpendicular to the main trunk of the left gastric artery. Patency of the left gastric artery was studied before and after surgery using Doppler ultrasound.

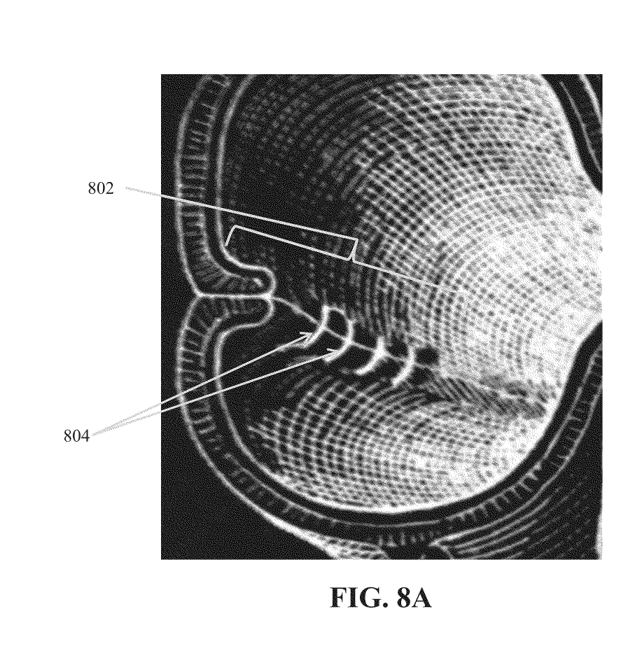

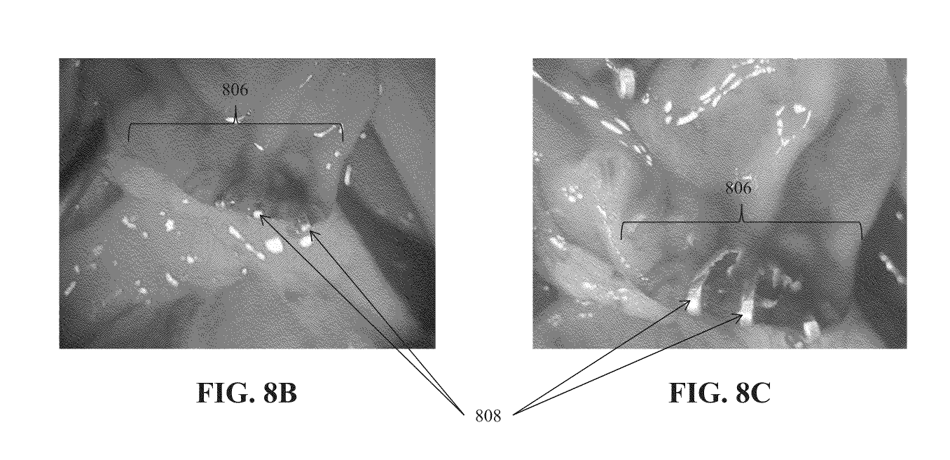

FIG. 8A is a diagram depicting an exemplary invagination of the fundus of the stomach of a subject rat. As shown in FIG. 8A, a portion of the fundus 802 is invaginated using clips (e.g., such as those denoted by reference number 804). FIGS. 8B and 8C are images of the fundus of the stomach of a subject rat, after invagination and clipping was performed. As shown in FIGS. 8B and 8C, a portion of the fundus 806 is invaginated and secured using clips (e.g., such as those denoted by reference number 808).

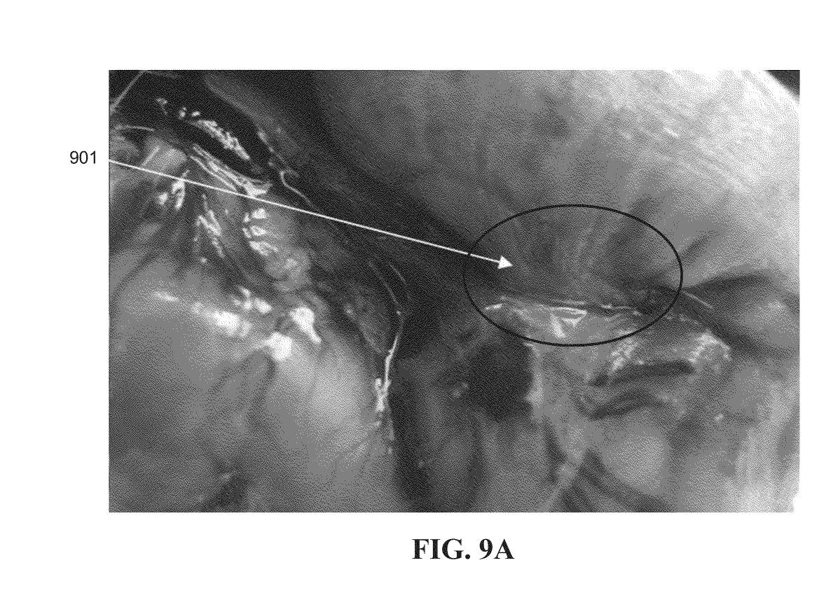

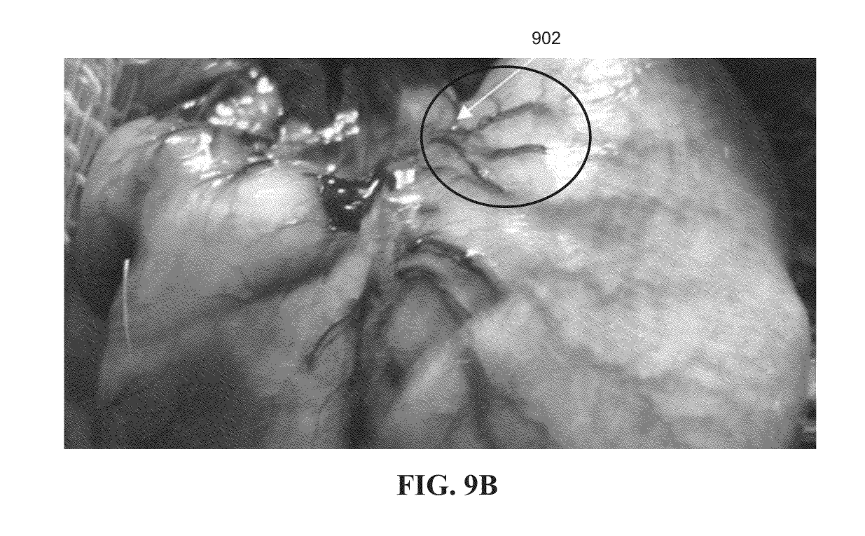

FIG. 9A is an image of the left gastric artery of the stomach of a subject rat, taken after the folding and clipping procedure was performed. As shown in FIG. 9A, the folded portion of the fundus appears in the circled area 901 and there is an absence of dark lines in proximity to the fold, indicating a reduction or interruption in blood flow.

In contrast, FIG. 9B is an image of the left gastric artery of the stomach of a subject rat, taken before the folding and clipping procedure was performed. As shown in FIG. 9B, several dark lines indicating operative blood vessels appear in the circled area 902 near the left gastric artery.

For the first treatment group of rats (in which ligation of the left gastric artery was performed), disruption of blood flow in the left gastric artery was achieved in 100% of the animals (as observed by visual inspection). For the second treatment group of rats (in which inward folding and clipping was performed), disruption of blood flow in the left gastric artery was achieved in 90% of the animals (as observed by visual inspection). For the control group, no disruption of blood flow was observed.

For the histopathology of the treated vessels taken from the rats post-mortem, tissue sections (5 mm thick) were cut from paraffin-embedded blocks on a microtome and mounted from warm water (40° C.) onto adhesive microscope slides and allowed to dry overnight at room temperature. Contiguous tissue sections were processed for standard hematoxylin-eosin staining After 3-5 days from clipping, the histological studies have shown the increase of leukocytes and cell elements in the mucous layer. Also, the hyperplasia of the mucous-forming cells was observed. Under the mucous layer, the widened capillaries with the blood stasis were observed. In some of the areas, the capillaries were damaged and they were not able to form the capillary network. Furthermore, the widening of the lymphatic capillaries were similar; they began to merge together with no particular pattern with different angles, and formed the closed loops establishing the capillary network.

CONCLUSIONS: Selectively clipping the gastric fundus to modify blood flow to the fundus as described in the study is feasible and appears to be safe. It leads to a reduction in plasma ghrelin levels at intermediate term follow-up. It may be a good tool to enhance weight loss in subjects with morbid obesity who cannot achieve weight loss by conventional means (diet and exercise) and an alternative to or complimentary to bariatric surgery.

Although the experiments above were performed in pigs and rats, it is expected that the inventive techniques described herein can be performed on humans to achieve similar results, due to the anatomical similarities between a pig's gastric system and a human's gastric system.

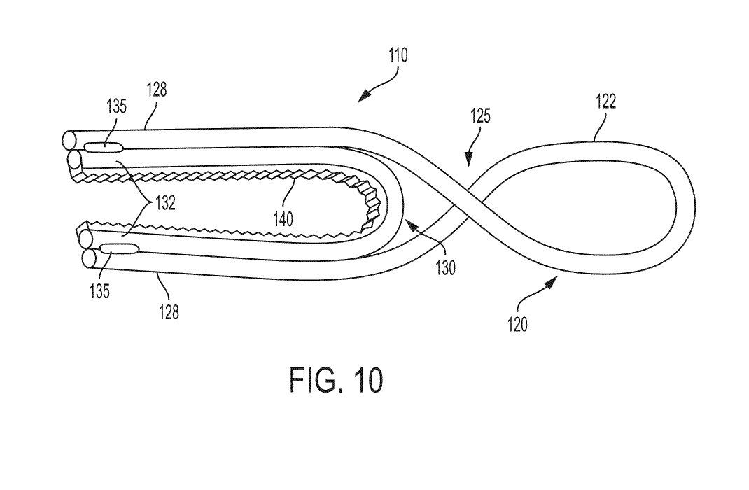

FIG. 10 depicts an example of a clip 110 that is suitable for performing clipping in human subjects. The clip 110 includes a first section 120 with an inversion region 125 disposed between a proximal portion 122 and a set of distal jaws 128. The first section 120 is configured so that when the proximal portion 122 is squeezed together, the distal jaws 128 will be forced apart. When the proximal portion is released, spring action causes the distal jaws to automatically return towards each other. Preferably, first section 120 is configured from spring material that springs back to the shape depicted in FIG. 10. Suitable materials for the first section 120 include titanium and spring-tempered stainless steel.

The clip 110 preferably also includes a U-shaped second section 130 positioned with the open end of the U facing in the distal direction. The distal end 132 of each arm of the second section 130 is preferably connected to the distal end of a respective one of the distal jaws 128, e.g., by welds 135. In alternative embodiments (not shown), an appropriate adhesive or an appropriate fastener is used in place of the welds 135 to connect the distal end 132 of each arm to the distal end of a respective one of the distal jaws 128. The U-shaped second section 130 is preferably made from a shape memory material (e.g., nitinol) that is configured to exert force in order to return to its original U shape, which provides additional clamping strength. In alternative embodiments, the U-shaped second section 130 is made from a spring material (e.g., spring-tempered stainless steel) that springs back to its original U shape. In alternative embodiments, either the first section 120 or the U-shaped second section 130 may be made from other materials e.g., a shape-memory polymer, polycarbonate, acrylonitrile butadiene styrene (ABS) or a resin.

In some embodiments, a ridged surface 140 is provided at the inner edge of the second section 130 (i.e., the side that contacts the fundus) to enhance the stability of the clip's position on the fundus. In the illustrated embodiment, the ridged surface 140 is implemented by affixing an additional piece of material to the second section 130. A hard material (e.g., metal or ceramic) may be used for this purpose, or alternatively a softer compressible material may be to minimize damage and trauma to the tissue. Examples of such softer materials include low-durometer polyurethane or sponge, silicone rubbers, or polymer gels. In alternative embodiments (not shown), a smooth or textured piece of any of the materials noted above may be used in place of the material with the ridged surface 140. In other alternative embodiments (not shown), the ridged surface 140 and the second section 130 may be integrally formed from a single piece of metal.

Optionally, the clip may be is coated with a substance to enhance a weight loss effect. Examples include, but are not limited to pharmaceuticals, genetic materials, different types of cells that also help to decrease production of ghrelin and/or other hormones, or other substances that effect appetite in humans.

To use this clip 110, the target region of the fundus is folded, and the proximal portion 122 of the clip 110 is squeezed together, which causes the jaws 128 to open. The clip is then pushed onto the folded portion of the fundus and the proximal portion 122 of the clip 110 is released. The spring action of the first section 120 and the force exerted by the shape memory of the U-shaped second section combine to urge the clip 110 to returns to its original shape (i.e., to return to the shape depicted in FIG. 10), resulting in the jaws 128 grasping the fundus and the clip 110 being secured in place. Note that the U-shape that is used in section 130 is advantageous because the wall of the human stomach is relatively thick, which can make it difficult to attach a conventional clips (in which the jaws are closer together when they are closed) to the stomach wall.

Suitable dimensions for the clip 110 are as follows: The overall length of the clip 110 is preferably between 4.5 and 5.5 cm, and most preferably about 5.5 cm. The length of the distal jaws 128 is preferably between 3 and 5 cm, and most preferably about 4 cm long. The width of the clip 110 is preferably between 2.5 and 3.5 mm. The spacing between the jaws in the closed position (depicted in FIG. 10) is preferably between 10 and 15 mm, and most preferably about 13 mm. The spacing between the jaws in the open position (not shown) is preferably between 13 and 19 mm, and most preferably about 16 mm. The amount of force needed to open the jaws is preferably 800±150 g, the closing strength which closes the clip is preferably between 400 and 500 g, a large portion of which is attributable to the use of memory metal in the U-shaped second section 130.

Although the methods and processes described herein are explained in the context of an example involving the left gastric artery and the fundus of the stomach, similar methods may be implemented in other portions of the stomach. Examples include, but are not limited to, portions of the stomach that are adjacent to the right gastric artery, the short gastric artery, the gastro-epiploic artery, the right gastro-omental artery, the left gastro-omental artery, or any branches thereof. In these alternative anatomic regions, the same apparatus and methods described above may be used. Alternatively, appropriate modifications may be made to optimize the apparatus and methods to the other portions of the stomach.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

We claim:

1. A method of promoting weight loss, the method comprising the steps of:
folding a healthy and non-bleeding portion of a mammal subject's gastric fundus in proximity to the distal portion of the subject's left gastric artery; and
securing the folded portion of the subject's gastric fundus using at least one fastener, wherein the at least one fastener is configured to squeeze the folded portion of the subject's gastric fundus with sufficient force to result in a reduction or interruption of blood flow to a portion of the gastric fundus that is ordinarily supplied with blood by the left gastric artery.

2. The method of claim 1, wherein the mammal subject is a human subject.

3. The method of claim 1, wherein the at least one fastener is configured to promote thrombosis in the vicinity of the distal portion of the subject's left gastric artery.

4. The method of claim 1, wherein the folding step comprises inwardly folding the portion of the subject's gastric fundus.

5. The method of claim 1, wherein the folding step is implemented using endoscopic pliers.

6. The method of claim 1, wherein the at least one fastener comprises a plurality of clips.

7. The method of claim 6, wherein the plurality of clips are positioned between 1 and 1.5 cm apart from each other.

8. The method of claim 1, wherein the at least one fastener includes a coating that comprises at least one of (a) a pharmaceutical that helps to decrease production of ghrelin, (b) a genetic material that helps to decrease production of ghrelin, and (c) cells that help to decrease production of ghrelin.

9. The method of claim 1, wherein the at least one fastener includes a coating that comprises a drug that helps to decrease or abolish inflammation around the at least one fastener.

10. The method of claim 1, wherein the mammal subject is a human subject, wherein the folding step comprises inwardly folding the portion of the subject's gastric fundus, and wherein the at least one fastener comprises a plurality of clips.

11. A method of modifying blood flow to a portion of a mammal subject's stomach that is ordinarily supplied with blood by a given artery, the method comprising the steps of:
folding a healthy and non-bleeding portion of a mammal subject's stomach in proximity to the given artery; and
securing the folded portion of the subject's stomach using at least one fastener, wherein the at least one fastener is configured to squeeze the folded portion of the subject's stomach with sufficient force to result in a reduction or interruption of blood flow to the portion of the subject's stomach that is ordinarily supplied with blood by the given artery.

12. The method of claim 11, wherein the mammal subject is a human subject.

13. The method of claim 11, wherein the folding step comprises inwardly folding the portion of the subject's stomach.

14. The method of claim 11, wherein the at least one fastener comprises a plurality of clips.

15. The method of claim 14, wherein the plurality of clips are positioned between 1 and 1.5 cm apart from each other.

16. The method of claim 11, wherein the given artery comprises at least one of a right gastric artery, a short gastric artery, a gastro-epiploic artery, right gastro-omental artery, a left gastro-omental artery, and any branches thereof.

17. The method of claim 16, wherein the mammal subject is a human subject, wherein the folding step comprises inwardly folding the portion of the subject's stomach, and wherein the at least one fastener comprises a plurality of clips.

18. A clip for clipping a portion of a human stomach, the clip comprising:
a first section having a proximal portion and a set of distal jaws, with an inversion region disposed between the proximal portion and the set of distal jaws, wherein the first section is configured from a spring material so that when the proximal portion is squeezed together, the distal jaws are forced apart, and that when the proximal portion is released, the distal jaws automatically return towards other by spring action; and
a U-shaped section having two arms, with a distal end of each arm connected to a distal end of a respective one of the distal jaws, wherein the U-shaped section is configured to exert force in order to return to its original U shape.

19. The clip of claim 18, further comprising a ridged surface provided at an inner edge of the U shaped section.

20. The clip of claim 18, wherein the U-shaped section is made from a shape memory material.

21. The clip of claim 18, wherein the clip has an overall length between 4.5 and 5.5 cm, wherein the distal jaws have a length between 3 and 5 cm.

22. The clip of claim 18, wherein the jaws are spaced between 10 and 15 mm apart in a closed position and wherein the jaws are spaced between 13 and 19 mm apart in an open position

23. The clip of claim 18, wherein the first section comprises at least one of titanium and stainless steel, and wherein the U-shaped section comprises nitinol.

24. The clip of claim 18, further comprising a ridged surface provided at an inner edge of the U shaped section, wherein the clip has an overall length between 4.5 and 5.5 cm, wherein the distal jaws have a length between 3 and 5 cm, wherein the jaws are spaced between 10 and 15 mm apart in a closed position, wherein the jaws are spaced between 13 and 19 mm apart in an open position, wherein the first section comprises at least one of titanium and stainless steel, and wherein the U-shaped section comprises nitinol.
